(19) United States
(12) Patent Application Publication
(10) Pub. No.: US 2021/0137849 A1
(43) Pub. Date: May 13, 2021

(54) ADHESIVE SKIN PATCH AND METHOD FOR MANUFACTURING THE SAME

(71) Applicant: Min Young BHANG, Seoul (KR)

(72) Inventor: Min Young BHANG, Seoul (KR)

(21) Appl. No.: 16/609,677

(22) PCT Filed: Apr. 19, 2019
(86) PCT No.: PCT/KR2019/004781

§ 371 Date: Oct. 30, 2019

(51) Int. Cl. A61K 9/70; A61K 33/00; A61K 47/34; A61N 2/00; A61K 47/32

(52) U.S. Cl. CPC A61K 9/7084; A61K 33/00; A61K 47/32; A61N 2/008; A61K 47/34

(57) ABSTRACT

Disclosed are an adhesive skin patch and a method for manufacturing the same. The adhesive skin patch includes i) one or more magnets including one or more selected from the group consisting of neodymium, ferrite and alnico, and having an aluminum oxide layer formed on a surface thereof, ii) a patch body configured to accommodate the one or more magnets, iii) an adhesive layer configured to be adhered to the patch body, and iv) an adhesive pad configured to have one surface adhered to the adhesive layer and the other surface facing a direction opposite to the surface and adhered to the skin. The adhesive layer includes a silicone-based resin, a curing agent and a thermoplastic resin, and the thermoplastic resin includes one or more selected from the group consisting of polypropylene, polyethylene, an ethylene vinyl acetate copolymer, an ethylene acrylate copolymer and thermoplastic urethane.

### TECHNICAL FIELD

The present invention relates to an adhesive skin patch and a method for manufacturing the same. More particularly, it relates to an adhesive skin patch which is easily adhered to the skin so as to effectively relieve pain through magnetic fields and a method for manufacturing the same.

### BACKGROUND ART

The N pole of a magnet pushes blood in the human body, contracts cells and thus inhabits functions of the cells, and the S pole pulls blood, relaxes cells and thus activates functions of the cells. Here, blood contains hemoglobin containing iron. When hemoglobin has magnetism by sufficiently receiving magnetic fields, hemoglobin effectively transports oxygen and excretes waste. On the other hand, when hemoglobin lacks magnetic fields, hemoglobin cannot perform an original function thereof and thus causes various diseases. Main functions managing human body metabolism are executed using electricity and magnetism, and health is maintained through circulation of blood throughout the whole body while maintaining a balance between the positive pole and the negative pole. However, if magnetic fields are insufficient, diseases of the human body occur.

Conventional adhesive skin patches were manufactured by inserting magnets into a silicone member which is harmless to the human body. However, when they are used for a long time, the surfaces of the magnets may be corroded. Further, when the adhesive skin patches are used for a long period of time, the adhesive skin patches may be easily detached from the skin.

### RELATED ART DOCUMENT

### Patent Document

(Patent Document 1) Korean Patent Registration No. 0794778

### DISCLOSURE

### Technical Problem

Therefore, the present invention has been made in view of the above problems, and it is an object of the present invention to provide an adhesive skin patch which has excellent skin adhesion, is usable semi-permanently, exhibits excellent magnetic therapeutic effects, and is usable in the whole human body. It is another object of the present invention to provide a method for manufacturing an adhesive skin patch.

### Technical Solution

In accordance with an aspect of the present invention, the above and other objects can be accomplished by the provision of an adhesive skin patch configured to be adhered to a skin. The adhesive skin patch includes i) one or more magnets including one or more selected from the group consisting of neodymium, ferrite and alnico, and having an aluminum oxide layer formed on a surface thereof, ii) a patch body configured to accommodate the one or more magnets, iii) an adhesive layer configured to be adhered to the patch body, and iv) an adhesive pad configured to have one surface adhered to the adhesive layer and the other surface facing a direction opposite to the surface and adhered to the skin. The adhesive layer includes a silicone-based resin, a curing agent and a thermoplastic resin, and the thermoplastic resin includes one or more selected from the group consisting of polypropylene, polyethylene, an ethylene vinyl acetate copolymer, an ethylene acrylate copolymer and thermoplastic urethane.

In accordance with a further aspect of the present invention, there is provided an adhesive skin patch configured to be adhered to a skin. The adhesive skin patch includes i) a patch body, with which one or more selected from the group consisting of germanium, copper oxide and nanoceramic are dispersedly mixed, ii) an adhesive layer adhered to the patch body, and iii) an adhesive pad configured to have one surface adhered to the adhesive layer and the other surface facing a direction opposite to the surface and adhered to the skin. The adhesive layer includes a silicone-based resin, a curing agent and a thermoplastic resin, and the thermoplastic resin includes one or more selected from the group consisting of polypropylene, polyethylene, an ethylene vinyl acetate copolymer, an ethylene acrylate copolymer and thermoplastic urethane.

The ethylene acrylate copolymer may include one or more selected from the group consisting of ethylene methacrylate (EMA), ethylene ethyl acrylate (EEA), ethylene vinyl acetate (EBA) or ethylene acrylic acid (EAA). The adhesive layer may include 45 wt % to 50 wt % of the thermoplastic resin.

The one or more magnets may include a plurality of magnets, and the patch body may include i) first patch body parts in which the magnets are installed, and ii) second patch body parts located between the magnets. The second patch body parts and the first patch body parts may be alternately provided and interconnected, and a thickness of the first patch body parts may be greater than a thickness of the second patch body parts. One or more of the magnets may be formed to have a conical shape, and vertices of the one or more magnets may be exposed to an outside of the adhesive pad. The patch body may include one or more selected from the group consisting of silicone, thermoplastic polyurethane (TPU), styrene-ethylene-butadiene-styrene (SEBS) and poly-olefin-elastomer (POE).

In accordance with another aspect of the present invention, there is provided a method for manufacturing an adhesive skin patch, the method including i) preparing one or more magnets including one selected from the group consisting of neodymium, ferrite and alnico, and having an aluminum oxide layer formed on a surface thereof, ii) preparing a patch body including one or more materials selected from the group consisting of silicone, thermoplastic polyurethane (TPU), styrene-ethylene-butadiene-styrene (SEBS) and poly-olefin-elastomer (POE), and configured to surround the one or more magnets, iii) preparing a compressed sheet by mixing and heating a silicone-based resin, a curing agent and a thermoplastic resin, iv) adhering the compressed sheet to the patch body, and converting the compressed sheet into an adhesive layer by curing the compressed sheet by heating the patch body and the compressed sheet at a temperature of 170° C. to 190° C., and v) adhering a hydrogel layer to the adhesive layer.

In the preparing the patch body, the patch body may be prepared as a liquid molded body. The preparing the patch body may include i) preparing a patch body lower part including the one or more materials, ii) locating the one or more magnets on the patch body lower part, and iii) preparing a patch body upper part including the one or more materials, and preparing the patch body in which the patch body upper part and the patch body lower part are unified while covering the one or more magnets with the patch body upper part.

In accordance with yet another aspect of the present invention, there is provided a method for manufacturing an adhesive skin patch, the method including i) preparing a patch body in which 0.0001 to 1 parts by weight of germanium, copper oxide or nanoceramic is impregnated with 100 parts by weight of a silicone resin, ii) preparing a compressed sheet by mixing and heating a silicone-based resin, a curing agent and a thermoplastic resin, iii) adhering the compressed sheet to the patch body, and converting the compressed sheet into an adhesive layer by curing the compressed sheet by heating the patch body and the compressed sheet at a temperature of 170° C. to 190° C., and iv) adhering a hydrogel layer to the adhesive layer.

In the preparing the compressed sheet, the thermoplastic resin may include one or more selected from the group consisting of polypropylene, polyethylene, an ethylene vinyl acetate copolymer, an ethylene acrylate copolymer and thermoplastic urethane, and the compressed sheet may include 45 wt % to 50 wt % of the thermoplastic resin.

### Advantageous Effects

The adhesive skin patch has excellent skin adhesion, and may thus be adhered to any painful body part and effectively treat pain of the corresponding body part. Further, the adhesive skin patch is convenient to use, and employs permanent magnets and may thus maintain strong magnetism for a long period of time and be used semi-permanently. The adhesive skin patch, in which the magnets having strong magnetism are embedded in a soft silicone body, has excellent skin adhesion, causes no allergies, is usable semi-permanently, and exhibits excellent magnetic therapeutic effects.

### DESCRIPTION OF DRAWINGS

FIG. 1 is a schematic perspective view of an adhesive skin patch in accordance with a first embodiment of the present invention.

FIG. 2 is a schematic cross-sectional view of the adhesive skin patch, taken along line II-II of FIG. 1.

FIG. 3 is a schematic cross-sectional view of an adhesive skin patch in accordance with a second embodiment of the present invention.

FIG. 4 is a schematic cross-sectional view of an adhesive skin patch in accordance with a third embodiment of the present invention.

FIG. 5 is a schematic cross-sectional view of an adhesive skin patch in accordance with a fourth embodiment of the present invention.

FIG. 6 is a schematic cross-sectional view of an adhesive skin patch in accordance with a fifth embodiment of the present invention.

FIG. 7 is a schematic perspective view of an adhesive skin patch in accordance with a sixth embodiment of the present invention.

FIG. 8 is a schematic perspective view of an adhesive skin patch in accordance with a seventh embodiment of the present invention.

FIG. 9 is a schematic perspective view of an adhesive skin patch in accordance with an eighth embodiment of the present invention.

FIG. 10 is a schematic perspective view of an adhesive skin patch in accordance with a ninth embodiment of the present invention.

### BEST MODE

Hereinafter, it will be understood that technical terms used herein serve only to refer to specific embodiments and do not intend to limit the present invention. Singular expressions used herein encompass plural expressions, unless they have clearly different contextual meanings. In the following description of embodiments of the present invention, terms, such as “including”, etc., will be interpreted as indicating the presence of specific characteristics, regions, integers, steps, operations, elements and/or components stated in the description, and do not exclude the presence or addition of one or more other specific characteristics, regions, integers, steps, operations, elements, components and/or groups.

Even if they are defined differently, all terms used herein, including technical terms and scientific terms, may have the same meaning as generally understood by those skilled in the art. In addition, it will be understood that terms defined in generally used dictionaries have meanings coinciding with related technological documents and contents disclosed herein, and are not interpreted as having ideal or formal meanings unless stated otherwise.

Hereinafter reference will be made in detail to various embodiments of the present invention, examples of which are illustrated in the accompanying drawings and described below. While the invention will be described in conjunction with exemplary embodiments, it will be understood that the present description is not intended to limit the invention to the exemplary embodiments.

FIG. 1 schematically illustrates the exploded structure of an adhesive skin patch 100 in accordance with a first embodiment of the present invention. The exploded structure of the adhesive skin patch 100 of FIG. 1 serves only to exemplarily describe the present invention, and does not limit the present invention. Therefore, the structure of the adhesive skin patch 100 may be modified into other forms.

As shown in FIG. 1, the adhesive skin patch 100 includes magnets 10 (shown in a dotted line), a patch body 20, an adhesive layer 30 and an adhesive pad 40. In addition, the adhesive skin patch 100 may further include other elements, as needed.

The magnets 10 have a cylindrical shape. Otherwise, the magnets 10 may be manufactured to have other shapes. For example, the magnets 10 may have a circular shape or a bar shape.

As shown in FIG. 1, the patch body 20 may be manufactured to have a plurality of arms 201. The magnets 10 are embedded in the respective arms 201 of the patch body 20. Three arms 201 are radially uniformly separated from one another, and thus, the patch body 20 may be stably adhered to the skin. Although FIG. 1 illustrates that three magnets 10 are used, a larger number of magnets may be used.

The patch body 20 may be formed as a flat type or a curved type which fits the human body. The magnets 10 are respectively disposed within the arms 201 of a patch body lower part which is partially formed to have half the overall height of the patch body. Then, the magnets 10 are covered with a patch body upper part which is partially formed to have half the overall height of the patch body. Further, the magnets 10 are embedded in the patch body 20 by unifying the patch body upper part and the patch body lower part. Otherwise, the patch body 20 may be provided as a liquid molded body so as to surround the magnets 10. That is, the patch body 20 is molded while surrounding the magnets 10, and then cured.

The patch body 20 is formed of soft silicone, thermoplastic polyurethane (TPU), styrene-ethylene-butadiene-styrene (SEBS) or poly-olefin-elastomer (POE). Therefore, the patch body 20 is freely deformable and has elastic resiliency, thus being easily adhered to the skin. That is, conventional silicone bracelets having embedded magnets may be worn at the wrists or ankles only, and may not be applied to the waists, shoulders, backs, hips, thighs, joints, etc. However, the patch body 20, which is freely deformable and has elastic resiliency, may be applied to these regions.

Further, most conventional skin adhesive magnet patches are disposable products in which magnets are directly adhered to the skin and then a tape is attached thereto. In these products, the magnets directly contact the skin and may cause side effects, such as allergic reactions. Further, these disposable products may not be reused after a single usage. In contrast, the patch body 20 in accordance with the first embodiment of the present invention surrounds the magnets 10, and thereby, the magnets 10 do not directly contact the skin, thereby overcoming these side effects.

If the patch body 20 is formed of soft silicone, a curing agent may be mixed with a silicone-based resin. The curing agent is manufactured by mixing 45 wt % of silicone-based powder, silicone oil and silica. In addition, a small amount of a silicone-based coloring agent, for example, 0.5 wt % of the silicone-based coloring agent, may be added. If a large amount of the curing agent is used, a curing time may be shortened. For example, the curing time of 3 minutes at a temperature of 170° C. may be reduced to 2 minutes to 2 minutes 30 seconds. However, soot may be generated and pre-curing may be caused. Further, if the amount of silica included in the curing agent is increased, hardness of the adhesive layer 30 is increased. Therefore, the hardness of the adhesive layer 30 may be controlled by adjusting the content of silica. Liquid silicone may use platinum as the curing agent.

Hardness of the patch body 20 may be 50 or less. If the hardness of the patch body 20 is excessively high, the patch body 20 may not be easily bent. The hardness of the patch body 20 may be particularly 30 or less, and more particularly 20 or less. For example, the patch body 20 may be manufactured using a material having hardness of 5 to 15, and thus, even if the adhesive skin patch 100 is adhered to the curved skin, the adhesive skin patch 100 may be easily transformed so as to fit the curved skin. Accordingly, the adhesive skin patch 100 widens a contact area with the skin and may thus continuously maintain the adhesive state with the skin even if the skin moves.

The adhesive layer 30 adheres the patch body 20 and the adhesive pad 40 to each other, and thus connects the same to each other. If the patch body 20 is formed of silicone, the adhesive pad 40 is not easily adhered to the patch body 20 due to residual siloxane or silicone oil. As a result, if the adhesive skin patch 100 is used for a designated period of time, the patch body 20 may be peeled off from the adhesive pad 40. That is, the patch body 20 has low adhesive strength with other materials, and thus it is necessary to compensate for this drawback.

For example, if the adhesive pad 40 is adhered to the patch body 20 by coating the patch body 20 with a primer, the primer is peeled off from the patch body 20 formed of silicone. Further, since the adhesive skin patch 100 is adhered to the human body, an adhesive which is harmful to the human body may not be used to adhere the adhesive pad 40 and the patch body 20 to each other. Even if the patch body 20 is implemented to have adhesive force of 1000 gf/20 mm by applying a specific coating agent to the surface of the patch body 20, the surface of the patch body 20 is hardened, and thus, when the patch body 20 is continuously used, the surface of the patch body 20 is broken and thereby, if the adhesive pad 40 is replaced, it is difficult to adhere a new adhesive pad to the surface of the patch body 20.

In contrast, the adhesive layer 30 used in the first embodiment of the present invention is easily adhered to the patch body 20 and the adhesive pad 40. That is, even if a polymer resin having no adhesive force with other materials is used as a material of the patch body 20, the adhesive layer 30 may be easily adhered to the patch body 20, and adhesive force of the adhesive layer 30 with the adhesive pad 40 may be significantly improved. Therefore, the adhesive skin patch 100 which has excellent durability and is harmless to the human body may be provided.

The adhesive layer 30 may be manufactured by mixing a silicone-based resin, a curing agent and a thermoplastic resin. For example, the adhesive layer 30 is manufactured by mixing a thermoplastic resin with a mixture including 0.9 wt % of a curing agent and the balance of a silicone-based resin.

As the thermoplastic resin, polypropylene, polyethylene, an ethylene vinyl acetate copolymer, an ethylene acrylate copolymer, thermoplastic urethane or mixtures thereof may be used. Here, the ethylene acrylate copolymer includes ethylene methacrylate (EMA), ethylene ethyl acrylate (EEA), ethylene vinyl acetate (EBA) or ethylene acrylic acid (EAA).

The adhesive layer 30 may include 45 wt % to 50 wt % of the thermoplastic resin. The balance may be a mixture, or the mixture and other impurities. When the amount of the thermoplastic resin is within the above-described range, adhesive force between the adhesive layer 30 and the adhesive pad 40 may be properly maintained. The adhesive layer 30 may be manufactured through the following process.

That is, the silicone-based resin, the curing agent and the thermoplastic resin are mixed, heated and compressed, thereby producing a compressed sheet having a thickness of 1 mm or less. Then, the compressed sheet is adhered to the patch body 20, and is cured at a temperature of 170° C. to 190° C., thereby being cured. The compressed sheet may be adhered to the patch body 20 at a proper adhesive force within such a temperature range. The compressed sheet is converted into the adhesive layer 30. The adhesive layer 30 may be detachably adhered to the adhesive pad 40.

The adhesive pad 40 is provided as a hydrogel. The hydrogel does not cause skin irritation, has excellent adhesion to the skin and has flexibility sufficient to be easily adhered to the skin along a curved surface of the skin. The adhesive pad 40 maintains adhesive force thereof even if it is frequently attached to and detached from the skin. If the adhesive pad 40 is contaminated due to frequent use of the adhesive skin patch 100, the adhesive skin patch 100 may be used after the adhesive pad 40 is washed with water. Further, after the adhesive pad 40 is used for a designated period of time, the adhesive pad 40 may be detached from the adhesive layer 30 and then replaced with a new adhesive pad. The adhesive pad 40 may include a functional material, such as germanium, copper oxide, nanoceramic, or the like, or may use combinations thereof.

FIG. 2 schematically illustrates a cross-sectional structure of the adhesive skin patch 100, taken along line II-II of FIG. 1, parallel to the X-axis. The cross-sectional structure of the adhesive skin patch 100 of FIG. 2 serves only to exemplarily describe the present invention, and does not limit the present invention. Therefore, the cross-sectional structure of the adhesive skin patch 100 may be modified into other forms.

As shown in FIG. 2, the magnet 10 includes a magnet main body 101, an aluminum layer 103 and an aluminum oxide layer 105. The magnet main body 101 includes neodymium, ferrite, alnico or the like. Neodymium, which is a rare earth element having strong magnetic characteristics, improves blood circulation, muscle pain, various joint pains, headache, dizziness, etc., and improves concentration. Ferrite may be manufactured using a sintering method depending on powder metallurgy. Ferrite may include iron oxide as a main ingredient, and may additionally include barium or strontium. Alnico includes an alloy of iron, nickel, aluminum and cobalt. Alnico is sufficiently strong to maintain magnetism even if it is used at a high temperature of 500° C. to 600° C. Therefore, it is highly effective in pain treatment.

The aluminum layer 103 surrounds the magnet main body 101. The aluminum layer 103 covers the magnetic main body 101. Further, the aluminum oxide layer 105 is formed on the aluminum layer 103. That is, the aluminum oxide layer 105 is formed by oxidizing the surface of the aluminum layer 103. For example, the magnetic main body 101 having the aluminum layer 103 formed thereon is heated to a temperature of 200° C. to 800° C. More particularly, the magnetic main body 101 is heated at a temperature of 300° C. to 600° C., thus forming the aluminum oxide layer 105. As a result, the aluminum oxide layer 105 prevents the magnetic main body 101 and the patch body 10 from directly contacting each other, thereby being capable of preventing corrosion of the magnetic main body 101.

The aluminum oxide layer 105 is formed on the aluminum layer 103. That is, the aluminum layer 103 is formed by performing special coating using aluminum on the magnet main body 101, and the aluminum oxide layer 105 is formed by oxidizing the surface of the aluminum layer 103. Conventionally, magnets coated with nickel, copper or zinc were used. However, when the conventional adhesive skin patches are used for a designated period of time, the surfaces of the magnets were oxidized or corroded, were discolored, and thus had a bad appearance, and magnet insertion spaces were inflated due to leakage of siloxane and thus easily damaged. Meanwhile, if non-coated magnets are inserted into a silicone member, siloxane included in silicone reacts with the magnets, and thus the magnets are corroded. In the first embodiment of the present invention, the aluminum oxide layer 105 prevents the magnetic main body 101 from being easily corroded due to the patch body 20. Further, the aluminum layer 103 and the aluminum oxide layer 105 may be sequentially formed on each of magnet main bodies coated with nickel, copper or zinc.

The magnets 10 are embedded in the patch body 20. The adhesive layer 30 is located under the patch body 20 and adhered to the patch body 20. The adhesive pad 40 is located under the adhesive layer 30 and adhered to the adhesive layer 30. The adhesive pad 40 includes one surface 401 facing a positive direction of the Z-axis and the other surface 403 facing a negative direction of the Z-axis. Therefore, the surface 401 and the other surface 403 face opposite directions. The surface 401 is adhered to the adhesive layer 30, and the other surface 403 is adhered to the skin. Since the adhesive pad 40 is detachably adhered to the adhesive layer 30, the surface 401 may be contaminated if it is used frequently. Therefore, the adhesive pad 40 may be used after the surface 401 is washed with water, or may be replaced with a new adhesive pad. Further, it is necessary to periodically wash the other surface 403.

FIG. 3 schematically illustrates a cross-sectional structure of an adhesive skin patch 200 in accordance with a second embodiment of the present invention. The cross-sectional structure of the adhesive skin patch 200 of FIG. 3 serves only to exemplarily describe the present invention, and does not limit the present invention. Therefore, the cross-sectional structure of the adhesive skin patch may be modified into other forms. Further, since the cross-sectional structure of the adhesive skin patch 200 of FIG. 3 is similar to the cross-sectional structure of the adhesive skin patch 100 of FIG. 2, some parts in the second embodiment, which are substantially the same as those in the first embodiment, are denoted by the same reference numerals even though they are depicted in different drawings, and a detailed description thereof will thus be omitted because it is considered to be unnecessary.

As shown in FIG. 3, a patch main body 22 includes first patch main body parts 221 and second patch main body parts 223. The first patch main body parts 221 and the second patch main body parts 223 are alternately provided and interconnected. Magnets 10 are installed in the first patch main body parts 221. Since the magnets 10 are inserted into the first patch main body parts 221, a thickness t221 of the first patch main body parts 221 is greater than a thickness t223 of the second patch main body parts 223. If the adhesive skin patch 200 is adhered to the skin, a user may desire rapid treatment effects by allowing the magnets 10 to be in contact with a painful area. In this case, protruding parts 221a are checked with the naked eye, and thus, the positions of the magnets 10 may be confirmed and the magnets 10 may be directly adhered to the painful area.

FIG. 4 schematically illustrates a cross-sectional structure of an adhesive skin patch 300 in accordance with a third embodiment of the present invention. The cross-sectional structure of the adhesive skin patch 300 of FIG. 4 serves only to exemplarily describe the present invention, and does not limit the present invention. Therefore, the cross-sectional structure of the adhesive skin patch may be modified into other forms. Further, since the cross-sectional structure of the adhesive skin patch 300 of FIG. 4 is similar to the cross-sectional structure of the adhesive skin patch 100 of FIG. 2, some parts in the third embodiment, which are substantially the same as those in the first embodiment, are denoted by the same reference numerals even though they are depicted in different drawings, and a detailed description thereof will thus be omitted because it is considered to be unnecessary.

As shown in FIG. 4, magnets 13 have an inverted conical shape. In addition, the magnets 13 may be formed to have a circular or bar shape. The magnet 13 includes a magnet main body 131, an aluminum layer 132 and an aluminum oxide layer 135. Additionally, the magnet 13 may further include other elements. A vertex of the magnet 13 having the inverted conical shape faces the negative direction of the Z-axis, and a base of the magnet 13 faces the positive direction of the Z-axis. The vertices of the magnets 13 face the skin, and may thus optimize magnetic field emission and raise treatment effects to a painful area or the skin.

FIG. 5 schematically illustrates a cross-sectional structure of an adhesive skin patch 400 in accordance with a fourth embodiment of the present invention. The cross-sectional structure of the adhesive skin patch 400 of FIG. 5 serves only to exemplarily describe the present invention, and does not limit the present invention. Therefore, the cross-sectional structure of the adhesive skin patch may be modified into other forms. Further, since the cross-sectional structure of the adhesive skin patch 400 of FIG. 5 is similar to the cross-sectional structure of the adhesive skin patch 200 of FIG. 3 and the cross-sectional structure of the adhesive skin patch 300 of FIG. 4, some parts in the fourth embodiment, which are substantially the same as those in the second and third embodiments, are denoted by the same reference numerals even though they are depicted in different drawings, and a detailed description thereof will thus be omitted because it is considered to be unnecessary.

As shown in FIG. 5, magnets 13 are installed in first patch main body parts 221. Since the magnets 13 are inserted into the first patch main body parts 221, a thickness t221 of the first patch main body parts 221 is greater than a thickness t223 of second patch main body parts 223. If the adhesive skin patch 400 is adhered to the skin, a user may desire rapid treatment effects by allowing the magnets 13 to be in contact with a painful area.

FIG. 6 schematically illustrates a cross-sectional structure of an adhesive skin patch 500 in accordance with a fifth embodiment of the present invention. The cross-sectional structure of the adhesive skin patch 500 of FIG. 6 serves only to exemplarily describe the present invention, and does not limit the present invention. Therefore, the cross-sectional structure of the adhesive skin patch may be modified into other forms. Further, since the cross-sectional structure of the adhesive skin patch 500 of FIG. 6 is similar to the cross-sectional structure of the adhesive skin patch 400 of FIG. 5 and the cross-sectional structure of the adhesive skin patch 300 of FIG. 4, some parts in the fifth embodiment, which are substantially the same as those in the fourth embodiment, are denoted by the same reference numerals even though they are depicted in different drawings, and a detailed description thereof will thus be omitted because it is considered to be unnecessary.

Magnets 13 promote formation of collagen in a dermal layer of the skin due to high strength magnetic fields. The magnets 13 exhibit strong magnetic treatment effects, such as imparting elasticity to a base layer, the dermal layer and a muscular layer.

As shown in FIG. 6, a vertex 109 of the magnet 13 of a focal radiation type having a conical shape is exposed to the outside of an adhesive pad 40. The vertex 109 contacts the skin and serves as a pressure spot, thus performing acupressure treatment and being more effective in pain relief. Further, the vertex 109 may be formed to have a rounded shape. Here, a radius of curvature of the vertex 109 may be 1 mm to 3 mm. When the radius of curvature of the vertex 109 is excessively small, the vertex 109 is sharpened and may thus pierce the skin. Further, the radius of curvature of the vertex 109 may not be excessively increased due to the size limit of the magnet 13. Therefore, by adjusting the radius of curvature of the vertex 109 within the above-described range, the vertex 109 may properly apply acupressure treatment to the skin without damage to the skin.

FIG. 7 schematically illustrates an adhesive skin patch 600 in accordance with a sixth embodiment of the present invention. An enlarged view of FIG. 7 schematically illustrates a cross-sectional structure of the adhesive skin patch 600. This structure of the adhesive skin patch 600 serves only to exemplarily describe the present invention, and does not limit the present invention. Therefore, the structure of the adhesive skin patch may be modified into other forms.

The adhesive skin patch 600 is provided as a flat type or a type in consideration of the ergonomic aspect satisfying human body curves, so as to increase adhesion to the human body. Therefore, the adhesive skin patch 600 may be easily adhered to the human body, and be applied to all areas of the human body regardless of a specific area. Even if the adhesive skin patch 600 is repeatedly used for a long period of time, the adhesive skin patch 600 may be semi-permanently used without corrosion or damage.

As shown in FIG. 7, an opening 69 is formed in the adhesive skin patch 600. Therefore, the adhesive skin patch 600 may be freely bent into a desired shape using the opening 69, and then be used.

Further, as shown in the enlarged view of FIG. 7, the adhesive skin patch 600 includes a patch body 60, an adhesive layer 64 and an adhesive pad 66. The patch body 60 includes functional materials 62. In addition, the adhesive skin patch 600 may further include other elements. The functions of the patch body 60, the adhesive layer 64 and the adhesive pad 66 are the same as the above-described functions, and a detailed description thereof will thus be omitted.

The functional materials 62 generate magnetic fields which are beneficial to the human body. The functional materials 62 emit far-infrared light and anions which have various effects, such as antibiotic, antifungal, anti-inflammatory and antiviral effects. As a result, the functional materials have a good influence not only on the epidermal layer but also on the dermal layer of the skin, thus improving skin conditions, such skin elasticity, whitening, moisturization, etc.

Germanium, copper oxide or nanoceramic may be used as the functional materials 62. The functional materials 62 are dispersed and mixed with the patch body 60. That is, the patch body 60 with which the functional materials 62 are mixed is manufactured by electrospraying a germanium-dispersed aqueous solution, a nanoceramic-dispersed aqueous solution or a copper oxide precursor aqueous solution to the patch body 60, and then drying and curing the solution sprayed to the patch body 60. The patch body 60 may be formed of a silicone resin. That is, 0.0001 to 1 parts by weight of germanium, copper oxide or nanoceramic may be impregnated with 100 parts by weight of the silicone resin.

Here, germanium activates metabolism in the human body. Further, germanium contains selenium which is high-unit natural tocopherol, and thus prevents aging. That is, germanium regenerates only aged parts, and alkalifies a human's physical constitution. Further, germanium is effective in skin care and against adult diseases, such as high blood pressure, diabetes, neuralgia, chronic rheumatism, arthritis, angina, etc. Germanium may have an average particle size of 1 nm to 90 nm. When the average particle size of germanium is excessively small, it is difficult to process the particles of germanium, and when the average particle size of germanium is excessively large, it is difficult to mix the particles of germanium with the patch body 60.

Copper oxide in an ionized state may be mixed with the patch body 60. Copper oxide is an essential mineral which strengthens the skin. Copper oxide plays the most important role in a skin regeneration process, and promotes protein production in the skin. If insoluble copper oxide in a copper ion type is mixed with the patch body 60, copper oxide is very effective in antibiotic and antiviral effects and wound healing. Further, copper oxide has excellent antibiosis, electromagnetic wave shielding function, and cosmetic effect. If a user wears the patch body 60 with which copper oxide is mixed during sleep, the patch body 60 contacts a moisture membrane of the skin and thus emits copper ions, and promotes production of collagen, elastin and proteins in the skin, and thus increase elasticity of the skin.

Nanoceramic emits anions and far-infrared light. Nanoceramic has an average particle size which exceeds 0 nm and is 1000 nm or less, and may thus be easily mixed with the patch body 60. Silicon carbide, alumina, silica, zirconia-silica, ZnO, TiO₂, CaCO₃, titanium oxide, tantalum oxide (Ta₂O₅), MgO, BeO or the like may be used as nanoceramic. Silicon carbide may have an average particle size of 300 nm to 500 nm, alumina may have an average particle size of 500 nm to 1000 nm, silica may have an average particle size of 700 nm to 1500 nm, zirconia-silica may have an average particle size of 500 nm to 1000 nm, ZnO may have an average particle size of 500 nm to 1000 nm, TiO₂ may have an average particle size of 100 nm to 300 nm, and CaCO₃ may have an average particle size of 500 nm to 1000 nm.

FIG. 8 schematically illustrates an adhesive skin patch 700 in accordance with a seventh embodiment of the present invention. This structure of the adhesive skin patch 700 serves only to exemplarily describe the present invention, and does not limit the present invention. Therefore, the structure of the adhesive skin patch 700 may be modified into other forms. Since elements of the adhesive skin patch 700 of FIG. 8 are the same as those of the adhesive skin patch 600 of FIG. 7, except for the shape thereof, some parts in the seventh embodiment, which are substantially the same as those in the sixth embodiment, are denoted by the same reference numerals even though they are depicted in different drawings, and a detailed description thereof will thus be omitted because it is considered to be unnecessary.

As shown in FIG. 8, the adhesive skin patch 700 has a shape, the width of which is gradually increased in directions toward the edges thereof. Therefore, the center of the adhesive skin patch 700 has the smallest width. Thus, the adhesive skin patch 700 is suitable for the skin of a body part, such as a wrist, which requires rolling-up of the adhesive skin patch 700 so as to be adhered thereto. The adhesive skin patch 700 includes a patch body 70 with which functional materials 62 are mixed. In addition, the adhesive skin patch 700 may further include other elements.

FIG. 9 schematically illustrates an adhesive skin patch 800 in accordance with an eighth embodiment of the present invention. This structure of the adhesive skin patch 800 serves only to exemplarily describe the present invention, and does not limit the present invention. Therefore, the structure of the adhesive skin patch 800 may be modified into other forms. Since elements of the adhesive skin patch 800 of FIG. 9 are the same as those of the adhesive skin patch 600 of FIG. 7, except for the shape thereof, some parts in the eighth embodiment, which are substantially the same as those in the sixth embodiment, are denoted by the same reference numerals even though they are depicted in different drawings, and a detailed description thereof will thus be omitted because it is considered to be unnecessary.

As shown in FIG. 9, the adhesive skin patch 800 has a shape having edges at which diagonally-stretched branches cross each other. Further, the center of the adhesive skin patch 800 has an oval shape. Therefore, the adhesive skin patch 800 is adhered to a severely curved body part from which the adhesive skin patch 800 is easily detachable. The adhesive skin patch 800 includes a patch body 80 with which functional materials 62 are mixed. In addition, the adhesive skin patch 800 may further include other elements.

FIG. 10 schematically illustrates an adhesive skin patch 900 in accordance with a ninth embodiment of the present invention. This structure of the adhesive skin patch 900 serves only to exemplarily describe the present invention, and does not limit the present invention. Therefore, the structure of the adhesive skin patch 900 may be modified into other forms. Since elements of the adhesive skin patch 900 of FIG. 10 are the same as those of the adhesive skin patch 600 of FIG. 7, except for the shape thereof, some parts in the ninth embodiment, which are substantially the same as those in the sixth embodiment, are denoted by the same reference numerals even though they are depicted in different drawings, and a detailed description thereof will thus be omitted because it is considered to be unnecessary.

As shown in FIG. 10, the adhesive skin patch 900 has a rectangular shape overall, and notch-type recesses 901 are formed at vertices of the adhesive skin patch 900. Therefore, a user may easily attach and detach the adhesive skin patch 900 to and from the skin by grasping regions around the recesses 901. The adhesive skin patch 900 includes a patch body 90 with which functional materials 62 are mixed. In addition, the adhesive skin patch 900 may further include other elements.

Hereinafter, the present invention will be described in more detail through the following test examples. These test examples serve merely to exemplarily describe the present invention and are not intended to limit the present invention.

### TEXT EXAMPLE OF ADHESIVE FORCE OF ADHESIVE LAYER

A mixture of a silicone-based resin and 0.9 wt % of a curing agent (L101), and ethylene vinyl acetate (EVA) (2615 produced by Hanwha Chemical Co., a VA content of 26%, and MI of 1.5) were uniformly mixed by a mixer at a temperature of 110° C. Then, an acquired mixture was compressed, thus producing a sheet having a thickness of about 1 mm. One surface of the sheet was adhered to a silicone-based resin, with which 0.9 wt % of a curing agent (L101) is mixed, at room temperature, and was then cured at a temperature of 170° C. to 190° C. for 4 minutes. Thereafter, a hydrogel layer was adhered to the other surface of the sheet, and adhesive force between a silicone layer and an adhesive layer and adhesive force between the adhesive layer and the hydrogen layer were measured using an adhesion tester. The remaining details about the test could be easily understood by those skilled in the art to which the present invention pertains, and a detailed description thereof will thus be omitted.

### Text Example 1

55 wt % of the mixture and 45 wt % of EVA were used. The remaining test process was the same as that of the above-described test example.

### Text Example 2

70 wt % of the mixture and 30 wt % of EVA were used. The remaining test process was the same as that of the above-described test example.

### Text Example 3

50 wt % of the mixture and 50 wt % of EVA were used. The remaining test process was the same as that of the above-described test example.

### Text Example 4

30 wt % of the mixture and 70 wt % of EVA were used. The remaining test process was the same as that of the above-described test example.

### COMPARATIVE EXAMPLE OF ADHESIVE FORCE OF ADHESIVE LAYER

A resin alone was compressed, thus producing a sheet having a thickness of about 1 mm. One surface of the sheet was adhered to a silicone-based resin, with which 0.9 wt % of a curing agent (L101) is mixed, at room temperature for 30 seconds, and was then cooled. Thereafter, adhesive force between the silicone-based resin and the sheet, i.e., an adhesive layer, was measured using the adhesion tester. The remaining details about the test could be easily understood by those skilled in the art to which the present invention pertains, and a detailed description thereof will thus be omitted.

### Comparative Example 1

Low-density polyethylene (LDPE) was used as the resin. The remaining test process was the same as that of the above-described comparative example.

### Comparative Example 2

EVA was used as the resin. The remaining test process was the same as that of the above-described comparative example.

### Comparative Example 3

Poly-olefin-elastomer (POE) was used as the resin. The remaining test process was the same as that of the above-described comparative example.

### Comparative Example 4

Ethylene methacrylate (EMA) was used as the resin. The remaining test process was the same as that of the above-described comparative example.

### Comparative Example 5

Thermoplastic polyurethane (TPU) was used as the resin. The remaining test process was the same as that of the above-described comparative example.

### Comparative Example 6

A hot-melt film was used as the resin. The remaining test process was the same as that of the above-described comparative example.

Results of Adhesive Force Test of Adhesive Layers

Results of adhesive force measurement of the adhesive layers according to the above-described test examples 1 to 4 and comparative examples 1 to 6 are stated in Table 1 below.

TABLE 1

| | Silicone-based | |
|---|---|---|
| | resin layer/ | Adhesive layer/ |
| Test example/ | adhesive layer | hydrogel layer |
| comparative example | (gf/20 mm) | (gf/20 mm) |
| Test example 1 | 1300 | 1000 |
| Test example 2 | 1000 | 300 |
| Test example 3 | 900 | 1000 |
| Test example 4 | 500 | 1100 |
| Comparative example 1 | 10 or less | — |
| Comparative example 2 | 10 or less | — |
| Comparative example 3 | 10 or less | — |
| Comparative example 4 | 10 or less | — |
| Comparative example 5 | 10 or less | — |
| Comparative example 6 | 10 or less | — |

Adhesive force between the hydrogel layer and the skin is about 500 gf/20 mm. Therefore, only if adhesive force between the silicone-based resin layer and the adhesive layer is at least 900 gf/20 mm, a manufactured adhesive skin patch is not peeled off. However, in comparative examples 1 to 6, adhesive force between the silicone-based resin layer and the adhesive layer is only 10 gf/20 mm, and thus the above condition may not be satisfied. Therefore, if LDPE, EVA, POE, EMA, TPU or the hot-melt film is used alone, it is difficult to manufacture an adhesive skin patch having a desired strength. In contrast, in the above-described test examples 1 to 3, the silicone-based resin layer/the adhesive layer and the adhesive layer/the hydrogel layer having adhesive forces which are difficult to peel off from each other may be acquired. Therefore, if 50 wt % to 55 wt % of the mixture and 45 wt % to 50 wt % of EVA are mixed, an adhesive skin patch having adhesive force of a desired level may be manufactured. In addition to EVA, even if polypropylene, polyethylene, an ethylene vinyl acetate copolymer, an ethylene acrylate copolymer, or thermoplastic urethane is used, the same result may be acquired.

### TEXT EXAMPLE OF ADHESIVE FORCE OF ADHESIVE PAD

Adhesive force of a magnetic patch including an adhesive pad formed of hydrogel was measured. While repeating attachment and detachment of the magnetic patch acquired by adhering a silicone-based resin and a hydrogel layer to both surfaces of the adhesive layer manufactured according to the above-described test example 1, change in the adhesive force of the magnetic patch was measured. The remaining details about the test could be easily understood by those skilled in the art to which the invention pertains, and a detailed description thereof will thus be omitted.

### Test Example 5

While firstly attaching and detaching the magnetic patch to and from the skin, adhesive force of the magnetic patch was measured. The remaining test process was the same as that of the above-described test example.

### Test Example 6

While attaching and detaching the magnetic patch to and from the skin 10 times, adhesive force of the magnetic patch was measured. The remaining test process was the same as that of the above-described test example.

### Test Example 7

While attaching and detaching the magnetic patch to and from the skin 20 times, adhesive force of the magnetic patch was measured. The remaining test process was the same as that of the above-described test example.

### Test Example 8

After the magnetic patch was attached to and detached from the skin 20 times and then the contaminated surface of the hydrogel layer was removed by attaching a 3M scotch tape to the contaminated surface and then detaching the tape from the hydrogel layer, while again attaching and detaching the magnetic patch to and from the skin, adhesive force of the magnetic patch was measured. The remaining test process was the same as that of the above-described test example.

### Test Example 9

While again attaching and detaching the magnetic patch to and from the skin 5 times, adhesive force of the magnetic patch was measured. The remaining test process was the same as that of the above-described test example.

### Test Example 10

While again attaching and detaching the magnetic patch to and from the skin 10 times, adhesive force of the magnetic patch was measured. The remaining test process was the same as that of the above-described test example.

### COMPARATIVE EXAMPLE OF ADHESIVE FORCE OF ADHESIVE PAD

Adhesive force of a magnetic patch including an adhesive pad formed of a double-sided silicone tape was measured. While repeating attachment and detachment of the magnetic patch acquired by adhering a silicone-based resin and a double-sided silicone tape to both surfaces of the adhesive layer manufactured according to the above-described test example 1, change in the adhesive force of the magnetic patch was measured. The remaining details about the test could be easily understood by those skilled in the art to which the invention pertains, and a detailed description thereof will thus be omitted.

### Comparative Example 5

While firstly attaching and detaching the magnetic patch to and from the skin, adhesive force of the magnetic patch was measured. The remaining test process was the same as that of the above-described test example.

### Comparative Example 6

While attaching and detaching the magnetic patch to and from the skin 10 times, adhesive force of the magnetic patch was measured. The remaining test process was the same as that of the above-described test example.

### Comparative Example 7

While attaching and detaching the magnetic patch to and from the skin 20 times, adhesive force of the magnetic patch was measured. The remaining test process was the same as that of the above-described test example.

### Comparative Example 8

After the magnetic patch was attached to and detached from the skin 20 times and then the contaminated surface of the double-sided silicone tape was removed by attaching a 3M scotch tape to the contaminated surface and then detaching the tape from the double-sided silicone tape, while again attaching and detaching the magnetic patch to and from the skin, adhesive force of the magnetic patch was measured. The remaining test process was the same as that of the above-described test example.

### Comparative Example 9

While again attaching and detaching the magnetic patch to and from the skin 5 times, adhesive force of the magnetic patch was measured. The remaining test process was the same as that of the above-described test example.

### Comparative Example 10

While again attaching and detaching the magnetic patch to and from the skin 10 times, adhesive force of the magnetic patch was measured. The remaining test process was the same as that of the above-described test example.

Results of Adhesive Force Test of Adhesive Pads

Results of adhesive force measurement of the adhesive pads according to the above-described test examples 5 to 10 and comparative examples 5 to 10 are stated in Table 2 below. For reference, proper adhesive force to the skin is 100 gf/20 mm.

TABLE 2

| | Adhesive force | Comparative | Adhesive force |
|---|---|---|---|
| Test example | (gf/20 mm) | example | (gf/20 mm) |
| Test example 5 | 426 | Comparative | 443 |
| | | example 5 | |
| Test example 6 | 204 | Comparative | 58 |
| | | example 6 | |
| Test example 7 | 148 | Comparative | 41 |
| | | example 7 | |
| Test example 8 | 426 | Comparative | 108 |
| | | example 8 | |
| Test example 9 | 200 | Comparative | 50 |
| | | example 9 | |
| Test example 10 | 140 | Comparative | 42 |
| | | example 10 | |

As test results, if the hydrogel layer is used as the material of the adhesive pad, compared to use of the double-sided silicone tape, it may be confirmed that adhesive force of the adhesive pad is continuously maintained even when the adhesive pad is used multiple times. Particularly, as the adhesive pad is used multiple times, contamination of the adhesive pad is increased due to dead skin cells, and thus reduction in the adhesive force of the adhesive pad using the double-sided silicone tape was greater than that of the adhesive pad using the hydrogel layer. Further, as stated in comparative example 8, even after the contaminated surface of the adhesive pad was removed, the double-sided silicone tape did not restore adhesive force thereof to an original state thereof, and remained at an adhesive force level of about 25%. That is, the double-sided silicone tape was available about once. Therefore, it may be confirmed that hydrogel is proper as the material of the adhesive pad. Although the exemplary embodiments of the present invention have been disclosed for illustrative purposes, those skilled in the art will appreciate that various modifications, additions and substitutions are possible, without departing from the scope and spirit of the invention as disclosed in the accompanying claims.

1. An adhesive skin patch comprising:
an adhesive pad configured to have one surface adhered to the adhesive layer and the other surface facing a direction opposite to the surface and adhered to a skin,
wherein the adhesive layer comprises a silicone-based resin, a curing agent and a thermoplastic resin, and the thermoplastic resin comprises one or more selected from the group consisting of polypropylene, polyethylene, an ethylene vinyl acetate copolymer, an ethylene acrylate copolymer and thermoplastic urethane.

2. An adhesive skin patch configured to be adhered to a skin, comprising:
a patch body, with which one or more selected from the group consisting of germanium, copper oxide and nanoceramic are dispersedly mixed;
an adhesive layer adhered to the patch body; and
an adhesive pad configured to have surface adhered to the adhesive layer and the other surface facing a direction opposite to the surface and adhered to the skin,
wherein the adhesive layer comprises a silicone-based resin, a curing agent and a thermoplastic resin, and the thermoplastic resin comprises one or more selected from the group consisting of polypropylene, polyethylene, an ethylene vinyl acetate copolymer, an ethylene acrylate copolymer and thermoplastic urethane.

3. The adhesive skin patch according to claim 1, wherein the ethylene acrylate copolymer comprises one or more selected from the group consisting of ethylene methacrylate (EMA), ethylene ethyl acrylate (EEA), ethylene vinyl acetate (EBA) or ethylene acrylic acid (EAA).

4. The adhesive skin patch according to claim 1, wherein the adhesive layer comprises 45 wt % to 50 wt % of the thermoplastic resin.

5. The adhesive skin patch according to claim 1, wherein:
the one or more magnets comprise a plurality of magnets; and
the patch body comprises:
first patch body parts in which the magnets are installed; and
second patch body parts located between the magnets,
wherein the second patch body parts and the first patch body parts are alternately provided and interconnected, and a thickness of the first patch body parts is greater than a thickness of the second patch body parts.

6. The adhesive skin patch according to claim 5, wherein one or more of the magnets are formed to have a conical shape, and vertices of the one or more magnets are exposed to an outside of the adhesive pad.

7. The adhesive skin patch according to claim 1, wherein the patch body comprises one or more selected from the group consisting of silicone, thermoplastic polyurethane (TPU), styrene-ethylene-butadiene-styrene (SEBS) and poly-olefin-elastomer (POE).

8. A method for manufacturing an adhesive skin patch, the method comprising:
preparing one or more magnets comprising one selected from the group consisting of neodymium, ferrite and alnico and having an aluminum oxide layer formed on a surface thereof;
preparing a patch body comprising one or more materials selected from the group consisting of silicone, thermoplastic polyurethane (TPU), styrene-ethylene-butadiene-styrene (SEBS) and poly-olefin-elastomer (POE), and configured to surround the one or more magnets;
preparing a compressed sheet by mixing and heating a silicone-based resin, a curing agent and a thermoplastic resin;
adhering the compressed sheet to the patch body, and converting the compressed sheet into an adhesive layer by curing the compressed sheet by heating the patch body and the compressed sheet at a temperature of 170° C. to 190° C.; and
adhering a hydrogel layer to the adhesive layer.

9. The method according to claim 8, wherein, in the preparing the patch body, the patch body is prepared as a liquid molded body.

10. The method according to claim 8, wherein the preparing the patch body comprises:
preparing a patch body lower part comprising the one or more materials;
locating the one or more magnets on the patch body lower part; and
preparing a patch body upper part comprising the one or more materials, and preparing the patch body in which the patch body upper part and the patch body lower part are unified while covering the one or more magnets with the patch body upper part.

11-12. (canceled)

13. The adhesive skin patch according to claim 2, wherein the ethylene acrylate copolymer comprises one or more selected from the group consisting of ethylene methacrylate (EMA), ethylene ethyl acrylate (EEA), ethylene vinyl acetate (EBA) or ethylene acrylic acid (EAA).

14. The adhesive skin patch according to claim 2, wherein the adhesive layer comprises 45 wt % to 50 wt % of the thermoplastic resin.

15. The adhesive skin patch according to claim 2, wherein the patch body comprises one or more selected from the group consisting of silicone, thermoplastic polyurethane (TPU), styrene-ethylene-butadiene-styrene (SEBS) and poly-olefin-elastomer (POE).
